(19) United States
(12) Patent Application Publication
(10) Pub. No.: US 2026/0083744 A1
(43) Pub. Date: Mar. 26, 2026

(54) METHODS TO STRENGTHEN VISUAL PATHWAYS

(71) Applicant: Georgia Tech Research Corporation, Atlanta, GA (US)

(72) Inventors: Ming-Fai Fong, Atlanta, GA (US); Robert D. Lamprecht, Atlanta, GA (US)

(21) Appl. No.: 19/341,461

(22) Filed: Sep. 26, 2025

Related U.S. Application Data
(60) Provisional application No. 63/699,635, filed on Sep. 26, 2024.

(51) Int. Cl. A61K 31/529; A61K 31/662; A61P 27/02

(52) U.S. Cl. CPC A61K 31/529; A61K 31/662; A61P 27/02

(57) ABSTRACT

Disclosed herein are methods of treating visual impairment in a subject.

### RELATED APPLICATIONS

This application claims the benefit of priority to U.S. Provisional Application No. 63/699,635, filed Sep. 26, 2024, the entire contents of which is hereby incorporated by reference in its entirety.

### STATEMENT OF GOVERNMENT SUPPORT

This invention was made with government support under EY029326 and NS127725 awarded by the National Institutes of Health. The government has certain rights in the invention.

### BACKGROUND

There are numerous conditions, both genetic and environmental, that result in visual impairment involving less than optimal signaling between the eyes (or an eye) and visual cortex. For example, amblyopia generally arises during infancy and early childhood when inputs to the visual cortex from the two eyes are poorly balanced. Amblyopia is a prevalent form of visual disability that affects up to 4% of the population of the United States and higher in medically underserved areas. There are numerous causes of amblyopia, e.g., strabismus (misalignment of the eyes), anisometropia (asymmetric refraction), opacities and obstructions of one eye (e.g., cataract), myopia (near sightedness) or hyperopia (far sightedness) or astigmatism in one eye, or abatement of high spatial frequency vision in one eye as occurs with an opacity of optical media. Characteristics of amblyopia generally include poor spatial acuity in one eye, and an attendant loss of stereopsis. The current standard of care is to promote recovery of the weak amblyopic eye by temporarily patching or blurring the fellow eye. However, the effectiveness of patching or blurring is limited by poor compliance, reduction of vision in the patched eye, and variable outcomes that typically do not include recovery of binocularity. Additionally, if the amblyopia is severe, standard treatments are ineffective when initiated after age 10. In fact, patching or blurring of the fellow eye is ineffective in one-third of patients. Further, such treatments can be psychologically damaging. Accordingly, new methods for treating amblyopia are required.

### SUMMARY OF THE INVENTION

In some aspects, the present disclosure provides methods of treating visual impairment in a subject comprising administering an inhibitor of the ON signaling pathway to the subject.

In further aspects, the present disclosure provides methods of strengthening a visual pathway in a subject comprising administering an inhibitor of the ON signaling pathway to the subject.

In yet further aspects, the present disclosure provides methods of strengthening a visual pathway in a subject comprising administering an inhibitor of the ON signaling pathway to the subject.

### BRIEF DESCRIPTION OF THE DRAWINGS

FIG. 1A is a schematic depicting the differences between global vision loss and pathway-specific loss.

FIG. 1B is a schematic depicting how a pair of input spikes are processed to output spikes by the brain.

FIG. 1C is a schematic depicting how the loss of one input spike inputs effects the output spike.

FIG. 2 shows an exemplary method for determining if the administration of an agent may improve pathway-specific vision loss.

FIG. 3 is a set of bar charts showing the effect of the administration of L-2-amino-4-phosphonobutyric acid (APB) on off and on stimuli, as well as grating stimuli. Initially, after administration, APB decrease the VEP magnitude of the on, off, and grating stimuli. However, after 24 hours, the VEP magnitude of all three stimuli increased.

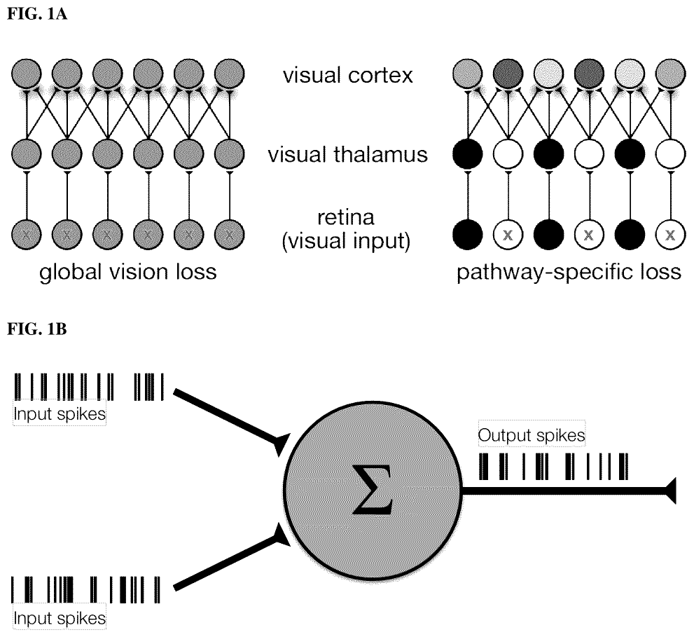

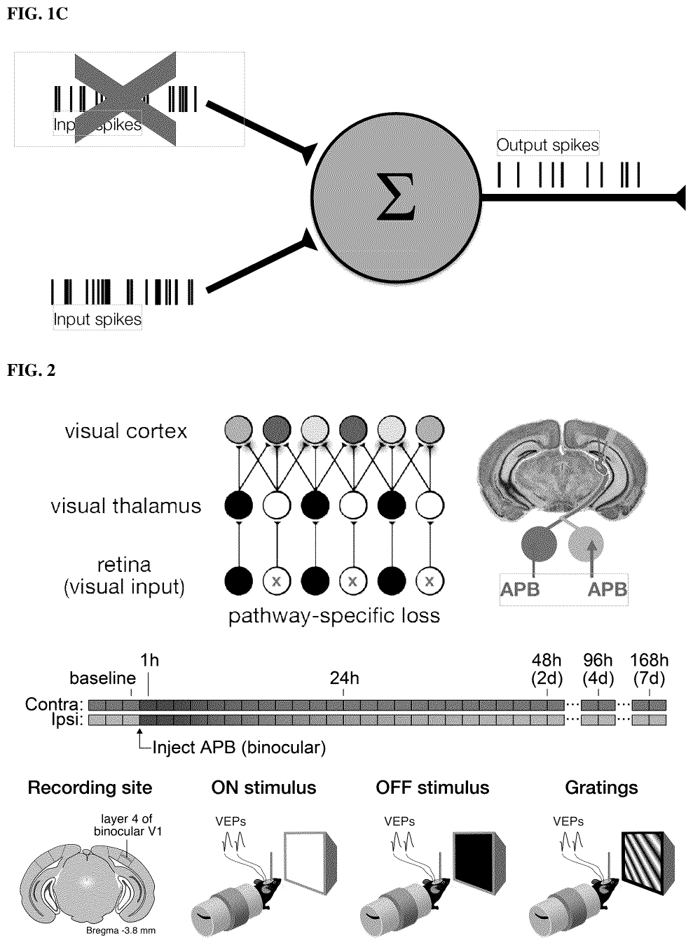

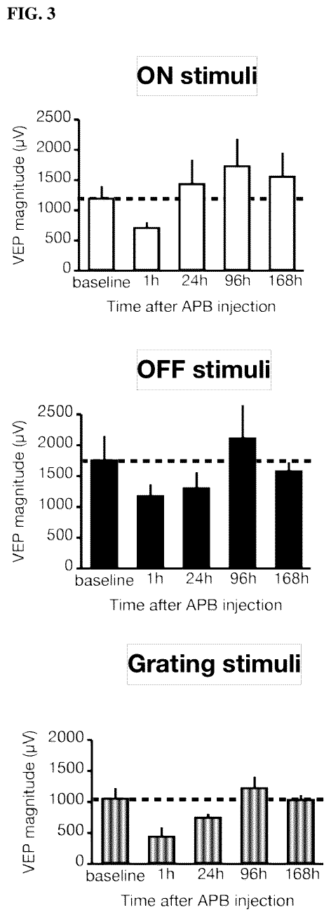

### DETAILED DESCRIPTION OF THE INVENTION

Early visual processing is broadly organized into two independent signaling pathways that respond to increments (ON) and decrements (OFF) in luminance. The ON and OFF signaling pathways emerge in the retina, operate in parallel through the thalamus, and combine within the primary visual cortex (V1). Transient bilateral inactivation of the retinas led to a long-lasting potentiation of visual cortical responses in mice. Visual enhancement can also be achieved via selective blockade of the ON retinal pathway. To inactivate the ON pathway, we used intravitreal injections of the mGlu6 antagonist, L-2-amino-4-phosphonobutyric acid (APB), to block depolarization of ON bipolar cells in both eyes. We also recorded visually-evoked potentials in the binocular zone of V1 before, during, and for several days after ON pathway blockade. Immediately after APB injection, we observed a pronounced disruption of visual responses across a range of visual stimuli. However, upon recovery of ON pathway signaling in subsequent days, we observed an increase in the magnitude of the visually-evoked responses to ON stimuli that exceeded baseline values; meanwhile responses to OFF and grating stimuli returned to baseline. While multiple forms of visual deprivation have been shown to drive homeostatic plasticity in V1, our observation that ON pathway blockade selectively drives potentiation of ON responses suggests that the compensatory response to visual deprivation may be pathway-specific.

In some aspects, the present disclosure provides methods of treating visual impairment in a subject comprising administering an inhibitor of the ON signaling pathway to the subject.

In certain embodiments, the inhibitor of the ON signaling pathway is an inhibitor of metabotropic glutamate receptor 6 (mGlu6). In certain embodiments, the inhibitor of the mGlu6 is an antagonist of mGlu6 or a negative allosteric modulator of mGlu6. In certain embodiments, the inhibitor of the ON signaling pathway is an inhibitor of transient receptor potential cation channel subfamily M member 1 (TRPM1). In certain embodiments, the inhibitor of the TRPM1 is an antagonist of TRPM1 or a negative allosteric modulator of TRPM1.

In certain embodiments, the inhibitor of the ON signaling pathway is L-2-amino-4-phosphonobutyric acid. In other embodiments, the inhibitor of the ON signaling pathway is tetrodotoxin.

In some aspects, the present disclosure provides methods of treating visual impairment in a subject comprising administering to the subject a means for inhibiting the ON signaling pathway.

In certain embodiments, the methods disclosed herein strengthens the subject's visual pathways (e.g., the ON pathway or the OFF pathway).

In certain embodiments, the visual impairment is ptosis, strabismus, anisometropia, childhood cataract, glaucoma, cloudy lens, or cloudy cornea. In other embodiments, the visual impairment is amblyopia. In certain embodiments, the visual impairment is myopia, glaucoma, retina degeneration, or amblyopia.

In some aspects, the present disclosure provides methods of strengthening a visual pathway in a subject comprising administering an inhibitor of the ON signaling pathway to the subject.

In certain embodiments, the visual pathways are located in the subject's primary visual cortex (e.g., V1). In certain embodiments, the visual pathway is the ON pathway. In certain embodiments, the visual pathway is the OFF pathway.

In certain embodiments, the inhibitors of the ON signaling pathway is administered via eye drops. In certain embodiments, the inhibitors of the ON signaling pathway is administered via intravitreal injection. In certain embodiments, the inhibitor of the ON signaling pathway is administered subretinally, epiretinally, or subconjunctivally.

In certain embodiments, the subject experiences visual improvement after about 24 hours, about 48 hours, about 96 hours, or about 168 hours after the inhibitor of the ON signaling pathway is administered to the subject. In certain embodiments, the subject experiences visual improvement after about 48 hours or about 96 hours.

In certain embodiments, the methods disclosed herein further comprise evaluating the subject's vision prior to administering the inhibitor of the ON signaling pathway.

In certain embodiments, the subject's visual improvement is measured relative to the subject's vision prior to administering the inhibitor of the ON signaling pathway

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W.H. Freeman & Co., New York (2000); Griffiths et al., “Introduction to Genetic Analysis, 7th ed.”, W.H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

### EXAMPLES

The invention now being generally described, it will be more readily understood by reference to the following examples which are included merely for purposes of illustration of certain aspects and embodiments of the present invention and are not intended to limit the invention.

### Example 1: Exemplary Treatment of Visual Impairment

It was demonstrated that transient inactivation of both retinas using tetrodotoxin (TTX) promotes recovery from deprivation amblyopia in mice and cats via homeostatic response potentiation. The study disclosed herein aimed to determine if selective inactivation of retinal cell subsets could replicate these effects. Given the disproportionate deficits in the ON pathway observed in humans with amblyopia, the study focused on selectively blocking ON signaling using L-2-amino-4-phosphonobutyric acid (APB).

### Methods

To monitor visual cortical activity longitudinally, tungsten microelectrodes were implanted into layer 4 of the primary visual cortex (V1) in normally-reared adolescent mice. Bilateral intravitreal injections of either TTX or APB were then performed and visually-evoked responses were assessed before, during, and several days post-pharmacological blockade. Full-field phase-reversing gratings and flash stimuli were utilized to evoke responses.

### Results

Full retinal inactivation with TTX produced a significant potentiation of visually-evoked cortical responses to grating stimuli just one day after injection, though this effect attenuated over the course of a week. In contrast, blockade of the ON pathway with APB initially weakened visual cortical responses to gratings but allowed recovery to baseline levels within a week. Notably, responses to ON stimuli relative to OFF stimuli were specifically elevated in APB-injected animals, suggesting pathway-specific homeostatic strengthening.

### Conclusion

Both partial and full retinal inactivation induce distinct forms of homeostatic plasticity in downstream visual circuits. The observed homeostatic shift in the balance of ON and OFF responses following selective ON pathway blockade suggests that targeted manipulation of visual pathways can offer therapeutic potential for amblyopia and other visual disorders involving imbalances in ON and OFF signaling.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of treating visual impairment in a subject comprising administering an inhibitor of the ON signaling pathway to the subject.

2. The method of claim 1, wherein the inhibitor of the ON signaling pathway is an inhibitor of metabotropic glutamate receptor 6 (mGlu6).

3. The method of claim 1, wherein the inhibitor of the mGlu6 is an antagonist of mGlu6 or a negative allosteric modulator of mGlu6.

4. The method of claim 1, wherein the inhibitor of the ON signaling pathway is an inhibitor of transient receptor potential cation channel subfamily M member 1 (TRPM1).

5. The method of claim 1, wherein the inhibitor of the TRPM1 is an antagonist of TRPM1 or a negative allosteric modulator of TRPM1.

6. The method of claim 1, wherein the inhibitor of the ON signaling pathway is L-2-amino-4-phosphonobutyric acid.

7. The method of claim 1, wherein the inhibitor of the ON signaling pathway is tetrodotoxin.

8. A method for treating visual impairment in a subject comprising administering to the subject a means for inhibiting the ON signaling pathway.

9. The method of claim 1, wherein the method strengthens the subject's visual pathways.

10. The method of claim 1, wherein the visual impairment is ptosis, strabismus, anisometropia, childhood cataract, glaucoma, cloudy lens, or cloudy cornea.

11. The method of claim 1, wherein the visual impairment is amblyopia.

12. The method of claim 1, wherein the visual impairment is myopia, glaucoma, retina degeneration, or amblyopia.

13. A method of strengthening a visual pathway in a subject comprising administering an inhibitor of the ON signaling pathway to the subject.

14. The method of claim 13, wherein the visual pathways are located in the subject's primary visual cortex.

15. The method of claim 13, wherein the visual pathway is the ON pathway.

16. The method of claim 13, wherein the visual pathway is the OFF pathway.

17. The method of claim 1, wherein the inhibitor of the ON signaling pathway is administered via eye drops.

19. The method of claim 1, wherein the inhibitor of the ON signaling pathway is administered via intravitreal injection.

20. The method of claim 1, wherein the inhibitor of the ON signaling pathway is administered subretinally, epiretinally, or subconjunctivally.

21-24.
